(19) United States
(12) Patent Application Publication
(10) Pub. No.: US 2011/0086926 A1
(43) Pub. Date: Apr. 14, 2011

(54) Disabling thymus gland as a new method for treatment of multiple sclerosis

(76) Inventor: Ali Nankali, Redhill (GB)

(21) Appl. No.: 12/925,098

(22) Filed: Aug. 24, 2009

(51) Int. Cl. A61K 45/00; A61B 17/00; A61P 37/00

(52) U.S. Cl. 514/789; 128/898

(57) ABSTRACT

This invention is related to the treatment of the Multiple Sclerosis (MS).

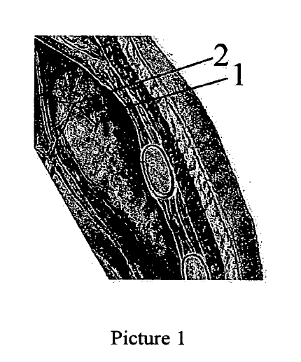

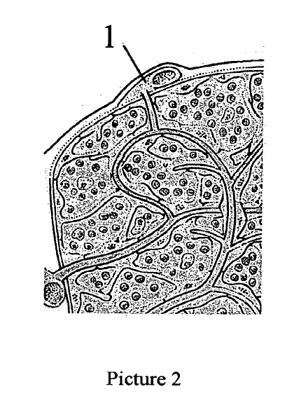

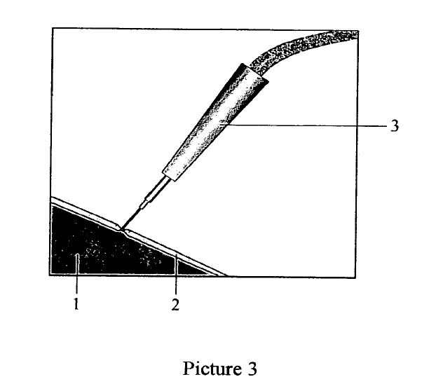

In this invention the disabling thymus gland is suggested as a new method of treatment for MS. This proposed method can be a surgical operation and or drug usage to achieve thymus hypofunction or dysfunction.

The idea is targeted on decreasing of the number of T-Cells by reaching a lower level of function of the Thymus Gland. These types of operation leads to stopping (partly or entirely) the maturation process of the blood stem cells to become a T-Cell.

Three ways are suggested to achieve the target:
1. Blocking the afferent arteries of thymus gland
2. Thymectomy
3. Using different drugs, which can dysfunction (partly or entirely) the thymus Gland

This method can be use as an additional method together with the other well-known methods.

### BACKGROUND

This invention is related to the treatment of the Multiple Sclerosis (MS).

In this invention the disabling thymus gland is suggested as a new method of treatment for Multiple Sclerosis.

It is well known that the MS is principally involved with the pathology of the Myelin Sheath with its different etiology such as: primary biochemical, abnormality of myelin exists and the processes which damages the myelin or oligodendrogial cell (demeylinating).

The research shows that the demyelinated areas are frequently under attack by T-cells and it has to be noticed that T-Cells effect damaged location in a different ways such as:
The toxins released from the accumulated T-cells do not allow the Myelin Sheath to have a physiological repair.
The released substances from the T-cells activate other T-Cells for attacking the pathological part of the Myelin Sheath.
Some released substances impact the T-cells to produce the specialized cells against the demeylinated area.

A huge effort is done to work out a solution to protect the Myelin Sheath from the T-Cells attacks. Although, the professionals were partly successful in their investigations on T-Cells and they found some solution to deactivate the released substances from T-Cells, block the process of releasing the toxins from T Cells and or provide an atmosphere in which T-Cells cannot reach the axons, there is no invented medicine or method of treatment to justify itself completely and therefore the T-Cells continue their attack to the Myelin Sheath.

Thymus is referred to a lymphoid organ that is located in the superior mediastinum and lower part of the neck and is necessary for the normal development of immunological function.

Thymectomy means surgical removal of the thymus gland. The thymectomy operation is not considered simple and it also leads to immune deficiency. Thymectomy is used for treatment Myasthenia gravis and it is becoming commonplace.

The afferent arteries are referred to the arteries that carry blood inward to a central organ or section.

Myelin Sheath is a segmented fatty lamination composed of myelin that wraps the axons of many nerves in the body. The usual thickness of the myelin sheath is between 200 and 800 μm. Various diseases such as multiple sclerosis can destroy myelin wrappings

T-Cells reffered to a principal type of white blood cell that completes maturation in the thymus and that has various roles in the immune system, including the identification of specific foreign antigens in the body and the activation and deactivation of other immune cells and it is also called T lymphocyte.

Etiology is the cause or origin of a disease or disorder as determined by medical diagnosis.

### STATEMENT OF INVENTION

The invention will now be described solely;

The invention is based on a completely different approach to treat the Multiple Sclerosis Disease for a first time. This proposed method can be a surgical operation and or drug usage to achieve thymus hypofunction or dysfunction.

The idea is targeted on decreasing of the number of T-Cells by reaching a lower level of function of the Thymus Gland in the body.

These types of operation leads to stopping (partly or entirely) the maturation process of the blood stem cells to become a T-Cell.

Three ways are suggested to achieve the target:
1. Blocking the afferent arteries of thymus gland
2. Thymectomy
3. Using different drugs, which can dysfunction (partly or entirely) the thymus Gland

### ADVANTAGES OF INVENTION

The invented method decreases (Partly or totally) the amount of T-Cells in the body.

The decreased amount of T-Cells reduces the quantity and quality of attacks to the myelin sheath by T-Cells.

The decreased amount of T-Cells reduces the quantity and quality of toxins realising by T-Cells.

The decreased amount of T-Cells reduces the quantity and quality of Specialised Cells targeting the myelin sheath.

The new proposed method of treatment created a condition in which specialists would be able to stimulate the Myelin Sheath repair or other significant processes by using a group of medicines that are already innovated.

### PICTURES

Picture one illustrates the thymus and its supplier-blood vessels and adjustment organs. 1—Thymus, 2—thymus blood vessels (Arteries and Veins)

Picture two illustrates the blood circulation in the thymus. 1—Artery

Picture three illustrates one method of blocking the afferent vessels using temperature: 1—Thymus, 2—afferent vessel, 3—burning instrument

I claim the ornamental for disabling Thymus Gland as a New Method for Treatment of Multiple Sclerosis as follow:

1. Thymectomy as a complete and or part of any method of treatment for Multiple Sclerosis.

2. Blocking the afferent arteries of thymus gland as a complete and or part of any method of treatment for Multiple Sclerosis.

3. Any Surgical technique related to the thymus as a complete and or part of any method of treatment of Multiple Sclerosis

4. Any surgery or operation on thymus gland as a complete and or part of any method of treatment for Multiple Sclerosis

5. Using any drug to disabling thymus gland as a complete and or part of any method of treatment of Multiple Sclerosis

6. Using any drug to achieve hypofunction Thymus as a complete and or part of any method of treatment of Multiple Sclerosis
